(19) United States
(12) Patent Application Publication
(10) Pub. No.: US 2017/0172530 A1
(43) Pub. Date: Jun. 22, 2017

(54) AN INTEGRATED DEVICE FOR CONDUCTING MAMMOGRAPHY, TOMOSYNTHESIS AND STEREOTACTIC BIOPSY IN MULTIPOSITION

(71) Applicant: PANACEA MEDICAL TECHNOLOGIES PVT LTD, Bangalore (IN)

(72) Inventors: G. v. Subrahmanyam, Bangalore (IN); P Manikandan, Bangalore (IN); Deepak Fernando, Bangalore (IN); Girish H, Bangalore (IN); Narayana Murthy H.M., Bangalore (IN); R. Afroza Khanam, Bangalore (IN); K. Kalyan Kumar, Bangalore (IN); D. Narasimha Reddy, Bangalore (IN)

(21) Appl. No.: 15/118,422

(22) PCT Filed: Jun. 20, 2014
(86) PCT No.: PCT/IB2014/062463

§ 371 Date: Aug. 11, 2016

(30) Foreign Application Priority Data
Feb. 14, 2014 (IN) 688CHE2014

(51) Int. Cl. A61B 6/00; A61B 6/06; A61B 6/04; A61B 6/02; A61B 10/02

(52) U.S. Cl. CPC A61B 6/502; A61B 6/025; A61B 10/0233; A61B 6/461; A61B 6/4447; A61B 6/04; A61B 6/06

(57) ABSTRACT

The invention discloses an integrated device for carrying out mammography, tomosynthesis and stereotactic biopsy of patient's breast in sitting, standing and lying down position. The patient's breast is positioned on the detector and x-ray beam of suitable energy is collimated on the patient's breast to take 2D, 3D images and perform stereotactic biopsy operations. The x-ray source and the detector plate is capable of moving at different angles with respect to each other that assist in taking images and perform stereotactic biopsy of patient's breast in sitting, standing and lying down position.

### TECHNICAL FIELD OF THE INVENTION

The present invention relates to an x-ray imaging device. In particular, the invention relates to an integrated device for carrying out mammography, tomosynthesis imaging and stereotactic biopsy that functions by selecting among a plurality of different imaging modes and different imaging angles. The device captures 2D and 3D images of patient's breast at sitting, standing and lying down position.

### BACKGROUND OF THE INVENTION

Nowadays breast cancer is the most common cancer among women's. Because of this wide spreading disease, early detection of tumour is highly significant. x-ray mammography has become the most commonly used tool for breast cancer screening, diagnosis and evaluation. A mammogram is an x-ray image of inner breast tissue that is used to visualize normal and abnormal structures within the breasts. Mammograms provide early cancer detection because they can often show breast lumps and calcifications before they are manually palpable.

While screening x-ray mammography is recognized as the most effective method for early detection of breast cancer, it also presents challenges in that in some cases it may be difficult to determine whether a detected abnormality is associated with a cancerous or benign lesion. One reason for this is that a mammogram is a two dimensional projection image representing a three dimensional structure, and overlapping structures in the compressed breast may confound image interpretation and diagnosis.

Efforts to improve the sensitivity and specificity of breast x-rays have included the development of breast tomosynthesis systems. Breast tomosynthesis is a three-dimensional imaging technology that involves acquiring images of a stationary compressed breast at multiple angles during a short scan. The individual projection tomosynthesis images taken at respective angles of the imaging x-ray beam relative to the breast are then computer-processed into a series of reconstructed tomosynthesis slice images each representing a respective slice of the breast.

In the prior art US20080045833A1 methods, devices, apparatuses and systems are disclosed for performing mammography, such as utilizing tomosynthesis in combination with breast biopsy. Utilizing these methods 3D coordinate of area of interest is computed using images of patient's tissue taken using tomosynthesis, and then needle is directed to the area of interest for performing biopsy. The above method fails to disclose a device that may perform mammography, tomosynthesis and stereotactic biopsy of a patient in sitting, standing and lying down position.

In an effort to address challenges in differentiating breast cancer from benign abnormalities in breast x-ray imaging it is highly desirable to design a device that may take x-ray images of the patient's breast in mammography and tomosynthesis mode and also assists in performing stereotactic biopsy. There is also a need of a device that may capture 2D and 3D image of patient's breast in multiposition such as sitting, standing and lying down position.

### SUMMARY OF THE INVENTION

According to an embodiment, the invention discloses an integrated device for carrying out mammography, tomosynthesis and stereotactic biopsy in sitting, standing and lying down position, the device consists of a compression pad and a detector for placing and compressing the patient's breast to be imaged, an x-ray tube assembly that contains x-ray source for generating x-rays of different energy that assist in taking 2D and 3D images of patient's breast and a display where the processed image of patient's breast is displayed. A mechanism by which 2D and 3D images of patient's breast will be taken at sitting, standing and lying down position. Multiposition image acquisition is achieved by capability of x-ray tube assembly to move at different angles with respect to detector where the patient's breast is kept to be imaged.

According to an embodiment, the x-ray tube assembly is rotatably mounted to gantry and can tilt in different angles to capture 2D and 3D image of patient's breast.

According to an embodiment, the pillar supporting the gantry is capable of moving vertically in up and down position and adjusts its height for positioning the patient comfortably in sitting, standing or lying down position.

### BRIEF DESCRIPTION OF DRAWINGS

The features of embodiments will become more apparent from the following detailed description of embodiments when read in conjunction with the accompanying drawings. Furthermore, elements that are known to be common and well understood to those in the industry are not depicted in order to provide a clear view of the various embodiments of the invention. Thus, in the interest of clarity and conciseness, the drawings are generalized in form.

FIG. 1 illustrates the components of a device for carrying out mammography, tomosynthesis and stereotactic biopsy in sitting, standing and lying down position in accordance with an embodiment of the invention.

### DETAILED DESCRIPTION OF THE INVENTION

Reference will now be made in detail to the description of the present subject matter, one or more examples of which are shown in figures. Each example is provided to explain the subject matter and not a limitation. Various changes and modifications obvious to one skilled in the art to which the invention pertains are deemed to be within the spirit, scope and contemplation of the invention.

The examples of device described in this patent specification leverage and combine advantages of one or more image acquisitions mode, including two-dimensional (2D), three-dimensional (3D) imaging to provide a breast imaging device with improved sensitivity and specificity and with benefits for more efficacious screening and diagnosis, greater convenience for patients.

The device according to one example includes an x-ray source including collimator jaws and multiple filters, an x-ray detector, a display, a mechanism to move the x-ray source and an adjustment mechanism between the x-ray source and the detector to place the patient's breast on the x-ray detector for taking images. During image acquisition, x-rays of two or more different energy ranges are generated from the x-ray source. The x-ray propagates through the patient's breast and is received by the detector. The composition of the patient's breast modulates the x-rays through mechanism such as attenuation, absorption, scatter resulting in relatively brighter and darker areas in the detected image. The detected image is further processed using computer processing techniques and the resulting image is displayed for further analysis and operation such as stereotactic biopsy. Utilizing the 2D and 3D images of patient's breast stereotactic biopsy is performed at a particular area of interest on the patient's breast.

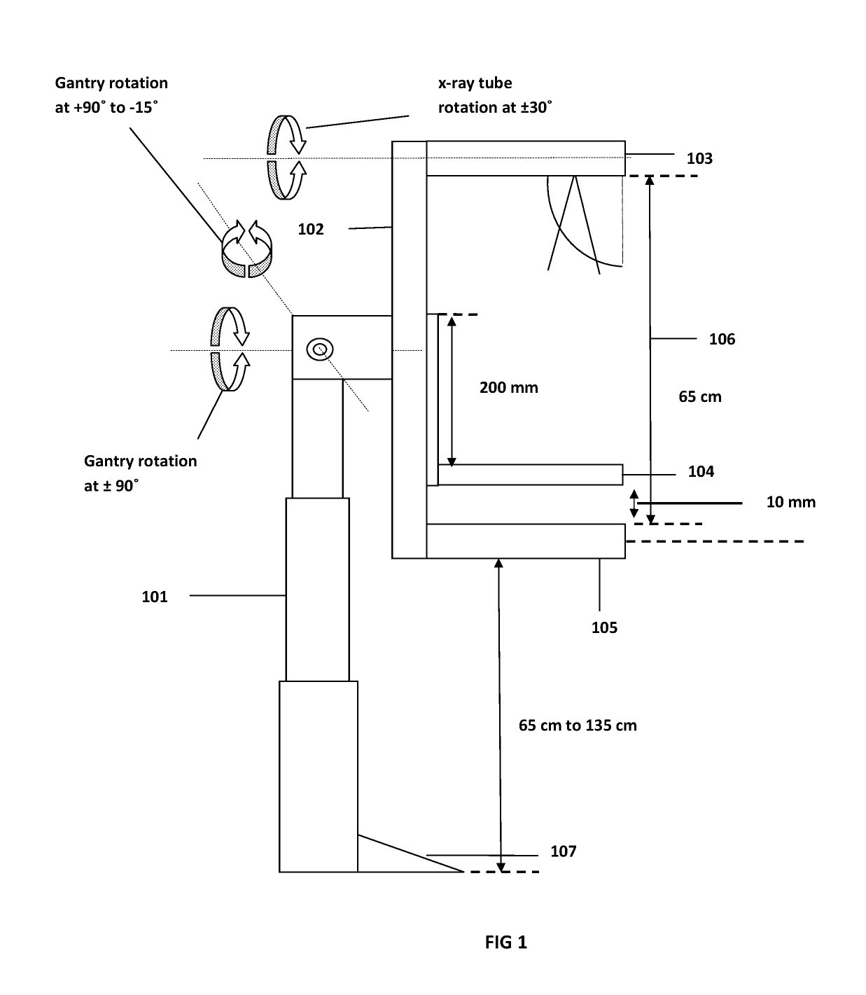

The device described in this patent specification is not limited to components and features described herein and may be modified according to requirements. FIG. 1 illustrates the components of a device for carrying out mammography, tomosynthesis and stereotactic biopsy in accordance with an embodiment of the invention. The device comprises a gantry (102) that is supported by a pillar (101). The pillar is capable of moving vertically in a range of 65 cm to 135 cm. This vertical motion is used to adjust the height for the patient to position the breast on the detector (105) either in standing, sitting or lying down position. The vertical motion takes place with a single constant speed selected automatically depending on the height of the patient. The gantry (102) is rotatably mounted to pillar (101) to pivot about a horizontal and vertical axis containing a housing supporting an x-ray tube assembly (102). The gantry (102) may be rotated at a rotation angle of ±90°. This gantry (102) movement is used to take 2D mammographic images in different angles. Some of the mammographic views captured using this gantry (102) movement will be Latero Medial (LM) view that will be image captured with the gantry (102) positioned at −90° and Medio Lateral (ML) view that will be image captured with the gantry positioned at +90°.

The gantry (102) can be tilted downward to −15° and tilted upwards to 90° where all the operations such as stereotactic biopsy may be performed with the patient lying on bed. This gantry (102) movement has an adjustment in between −15° to +15°; this is useful when patient is in sitting position. With the patient in this sitting position, 2D image, 3D image and biopsy operation may be achieved with ease. Some of the mammographic views captured using this gantry (102) movement will be Cranio Caudal (CC) view that will be image captured with the gantry (102) positioned at 0°, Left CC view that will be image captured with the gantry (102) positioned at −15°. Right CC that will be image captured with the gantry (102) positioned at 15°. Left Medio-Lateral Oblique (LMLO) that will be image captured with the gantry (102) positioned at −45°. Right Medio-Lateral Oblique (RMLO) that will be image captured with the gantry (102) positioned at 45°.

The x-ray tube assembly (103) is attached rotatably to the gantry (102) that may be rotated at a rotation angle of ±30° to acquire 3D images during tomosynthesis. For example for capturing 3D image of patient's breast the x-ray tube assembly (103) moves to 30° angle and starts x-ray firing at regular pulse and then moves towards −30°. Once it reaches −30° angle x-ray firing stops with buzzer and x-ray tube assembly (103) comes back to 0° and stops moving and releases the compression automatically. This may be performed with the gantry (102) positioned at any angle and with the patient positioned in standing or in sitting position. While positioning the patient's breast on the detector (105), the x-ray tube assembly (103) may be moved to an opposite end, so that the positioning of the patient's breast may be done with comfort, without bending the patient. The x-ray tube assembly (103) is supported by multiple x-ray filters and collimator jaws for selectively collimating the x-ray beam.

The collimator jaws present in x-ray tube assembly (103) collimates the x-ray beam on the patient's breast to be imaged in a field range of 23×29 cm. There are four additional filtration materials inside collimator that are 0.1 mm Rh, 0.1 mm Ag, 0.1 mm Mo, 0.5 mm Al all along with 1 mm Al added filter material. Filters are used to reduce the x-ray exposure. Filter material is automatically selected depending on the thickness of patient's breast and may be selected manually.

A compression pad (104) is mounted on housing of gantry (102) that is capable of moving vertically in a range of 200 mm. A detector (105) is attached to the housing of gantry (102) that serve as breast platform where the patient's breast to be imaged will be placed. The compression pad (104) moves up and down to accommodate the patient's breast to be imaged in between the compression pad (104) and detector (105) with a minimum distance of 10 mm. The mechanism by which the compression pad (104) moves is pneumatic. The compression pad (104) movement contains single speed, but releasing the compression when emergency button or decompression button pressed is done on high speed.

A face shield (106) is fixed to the x-ray tube assembly (103) that may be removed depending on the situation. The face shield is removed during carrying out tomosynthesis and remains fixed while performing other operations to provide a mechanical interlock against patient contact with the moving x-ray tube assembly (103). A display (107) is attached at the base of the pillar (101) where the 2D and 3D image of the patient's breast received from detector (105) is displayed.

The foregoing description of the specific embodiments will so fully reveal the general nature of the embodiments herein that others may, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein may be practiced with modification within the spirit and scope of the embodiments as described herein.

We claim:

1. An integrated device for carrying out Mammography, Tomosynthesis and Stereotactic biopsy in sitting, standing and lying down position, the device comprises:
a) a Gantry (102) comprising of Compression pad (104) and a Detector (105) for placing and compressing the patient's breast to be imaged;
b) an x-ray tube assembly (103) mounted in said Gantry (102) contains x-ray source for generating x-rays of different energy that assist in taking 2D and 3D images of patient's breast;
c) a display (107) where the processed image of patient's breast is displayed;
d) a Pillar (101) supporting said Gantry (102) that moves vertically in up and down motion to adjusts height of said device for positioning patient's at sitting, standing and lying down position.

2. The device as claimed in claim 1 wherein said Gantry (102) is capable of rotating at different angles for image acquisition in different modes and different positions.

3. The device as claimed in claim 1 wherein said x-ray tube assembly (103) will tilt in different angles and capture 2D and 3D image of patient's breast.

4. The device as claimed in claim 1 wherein said Compression pad (104) compresses the breast placed at Detector (105) to a minimum breast thickness of 10 mm.

5. The device as claimed in claim 1 wherein said Pillar (101) moves vertically in a travel range of 65 cm to 135 cm.

6. The device as claimed in claim 1 wherein said x-ray tube assembly (103) contains collimator for collimating the x-ray beams on the patient's breast to be imaged in a field size of 23×29 cm.

7. The device as claimed in claim 1 wherein said x-ray tube assembly (103) is attached to face shield (106) to provide a mechanical interlock against patient contact with the moving x-ray tube assembly.
